(19) United States
(12) Patent Application Publication
(10) Pub. No.: US 2023/0137481 A1
(43) Pub. Date: May 4, 2023

(54) Micro Incisional Manual Cataract Surgery

(71) Applicant: Badrudin Kurwa, Arcadia, CA (US)

(72) Inventor: Badrudin Kurwa, Arcadia, CA (US)

(21) Appl. No.: 17/974,151

(22) Filed: Oct. 26, 2022

Related U.S. Application Data
(60) Provisional application No. 63/274,003, filed on Oct. 31, 2021.

(51) Int. Cl. A61F 9/007

(52) U.S. Cl. CPC A61F 9/00745; A61F 2009/00887

(57) ABSTRACT

A portable rechargeable ultrasonic apparatus to remove a cataract nucleus includes an ultrasonic handpiece or body; an insulating sleeve, the insulating sleeve to cover a distal or far end of the ultrasonic handpiece or body; and a phaco cutter to connect to the ultrasonic handpiece or body. The phaco cutter includes a phaco cutter main body and a phaco cutter tip, wherein the phaco cutter tip is disposed at the distal end of the phaco cutter and has a width ranging from 2.75 to 3.25 millimeters.

### RELATED CASES

This application claims priority to application Ser. No. 62/274,003, filed Oct. 31, 2021, entitled “Micro Incisional Manual Cataract Surgery (MIMCS)”, the entirety of which is hereby incorporated by reference.

### BACKGROUND

In most of the technologically developed countries, the gold standard or best existing procedure for cataract surgery is the phacoemulsification (phaco) technique utilizing a sutureless 3 mm clear cornea incision. This technique can be performed with topical anesthesia with mild sedation and is much safer during the actual surgery because the wound is very small and self-sealing. Afterwards, the recovery from the surgery is faster, the risk of complications is far less and astigmatic changes to the patient's eyes are minimal. The disadvantage of utilizing this technique is the high cost of the phacoemulsification (phaco) device or apparatus in terms of acquisition cost, maintenance costs, repairs and, most importantly, the single use packs (including a phaco tip) needed for each patient case and is frequently unaffordable in the developing countries or in rural settings.

In addition, when utilizing the existing technique described above, there is also a small risk to the corneal endothelium when operating on dense cataracts. In these cases, a significant amount of ultrasonic energy is expended inside the eye in close proximity to the corneal endothelium. It is well known that even in the best use cases, there is an approximate 20% loss of endothelial cells. Such a loss of endothelial cells does not matter in healthy corneas as there are plenty of reserve cells. However, in patients with Fuchs dystrophy or early bullous keratopathy, there is a risk of corneal decompensation even requiring a corneal transplant in extreme cases.

In less technologically developed countries, phacoemulsification technology may be unavailable or unaffordable, or in patient cases where the risk to the cornea is unacceptable, an alternative technique or procedure that is used is the well-established Manual Extracapsular technique. The Manual Extracapsular technique requires up to a 10 mm scleral incision. Because of this the Manual Extracapsular technique has several disadvantages: 1) it requires retrobulbar anesthesia; 2) the surgery takes much longer; 3) cautery needs to be used to stop any bleeding; 4) sutures are needed to close the relatively large incision; 5) there are greater risks of intra operative complications; and 6) a risk of significant surgically induced astigmatism amongst others. One advantage of the Manual Extracapsular technique is that only an inexpensive intravenous line (IV) is needed for fluid irrigation to maintain the anterior chamber and an inexpensive aspiration cannula can be attached to a syringe for aspiration of the residual cortex.

Accordingly, a need exists for an economical affordable cataract removal technique that can be made available for eye surgeons around the world.

### BRIEF DESCRIPTION OF THE DRAWINGS

FIG. 1 illustrates a portable rechargeable ultrasonic cutting handpiece including a phaco cutter according to some embodiments;

FIG. 2A illustrates a front view of the phaco cutter according to some embodiments;

FIG. 2B illustrates a side view of the phaco cutter according to some embodiments;

FIG. 3A illustrates the created clear corneal wound according to some implementations;

FIG. 3B illustrates a phaco cutter on a cataract nucleus surface according to some implementations;

FIG. 3C illustrates a block diagram of a portable rechargeable ultrasonic cutting handpiece including a phaco cutter according to some embodiments; and

FIG. 4 illustrates a flowchart for performing the cataract surgical technique according to some embodiments.

### DETAILED DESCRIPTION

The following detailed description provides a better understanding of the features and advantages of the invention described in the present disclosure in accordance with the embodiments disclosed herein. Although the detailed description includes many specific embodiments, these are provided by way of example only and should not be construed as limiting the scope of the inventions disclosed herein.

The subject matter described herein includes a unique and novel apparatus and/or a unique and novel technique or process for cataract surgery. Described herein is a micro-incisional manual cataract surgical technique using ultrasound to divide the nucleus. In this surgical technique or process described herein, a surgeon makes a corneal limbal biplanar incision between 3 to 4 millimeters wide, which allows the incision to be self-sealing and sutureless and requires only topical anesthesia and mild sedation. In addition, this novel or new technique utilizes a portable handheld ultrasonic device that utilizes a minimal amount of ultrasonic energy only to divide the cataract nucleus into 3 easily removable longitudinal slivers. In some embodiments, the ultrasound generating device may be an inexpensive handheld device similar to a portable ultrasonic toothbrush that may be utilized to generate the ultrasound energy to divide the cataract nucleus into easily removable slivers using a specially designed cutter needle attached to it. Since the ultrasound is only used to divide the nucleus, corneal exposure to the ultrasonic energy is minimized. Since the nucleus is divided into three 3 mm wide longitudinal slivers these slivers can be removed from the eye with a 3 to 4 mm incision manually thus obviating the need for a large entry wound, the need for retrobulbar anesthesia, and the need for sutures. And most importantly it is quite inexpensive and affordable for eye surgeons to use all over the world.

The normal nucleus of the cataract is about 8 mm in diameter. In the illustrative embodiments, utilizing the unique process described herein, the cataract nucleus may be sliced into 3 longitudinal slivers or sections. In these illustrative embodiments, the 3 longitudinal slivers or sections can then be delivered easily through a 3 to 4 mm biplanar, self-sealing clear corneal limbal incision. In illustrative embodiments, the cataract nucleus may be divided using a portable ultrasonic device or apparatus (e.g., a modified ultrasonic toothbrush) to generate ultrasonic energy and a unique and novel cutting tip. In these illustrative embodiments, the portable ultrasonic device or apparatus tip may allow cutting the hardest of cataract nuclei into three longitudinal slivers or sections that can be delivered or removed through a 3 to 4 mm clear corneal incision.

In illustrative embodiments, an ultrasonic needle with a thin, flat blade shaped tip may be utilized to cut or make a narrow channel or incision in the hard anterior surface of the cataract nucleus using ultrasonic energy, much like using a thin flat screwdriver shaped cutting tip that is used to cut a narrow channel when cutting concrete. In order to make a hard surface easier to cut, in illustrative embodiments, a cutting tip with a narrow thickness is much more effective. The current standard Kelman phaco tip is 0.9 mm wide. The current 0.9 millimeter width is necessary because the opening at the apex of the Kelman phaco tip is used to aspirate fluid along with the emulsified cataract nuclear material to allow visualization of the anterior chamber of the eye during the cataract phacoemulsification step.

In illustrative embodiments, the phaco cutter described herein is designed only for cutting the nucleus and thus no aspiration is necessary. In these illustrative embodiments, the small amount of debris released from the cutting step is easily washed out of the wound by the irrigating fluid flowing through the anterior chamber of the eye from the inexpensive IV bottle without impeding the view. In illustrative embodiments, the distal end of the phaco cutter tip may be beveled to a sharp edge (like a knife) so it can cut into the hardest cataract nucleus with ease. In these illustrative embodiments, a width or thickness of the solid tip blade at the phaco cutting tip may be 0.3 to 0.4 mm thick instead of the current 0.9 mm. In these illustrative embodiments, by reducing the thickness of the phaco cutting tip by a factor of 3, this reduces the ultrasonic energy exposure by a factor of 9 as compared to the current 0.9 mm Kelman phaco tip in use. This is a significant improvement which results in energy savings as well as the benefits discussed previously.

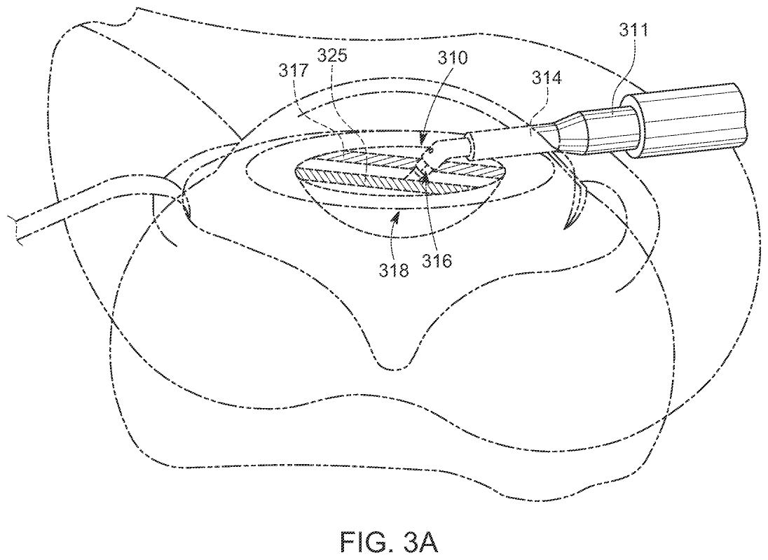

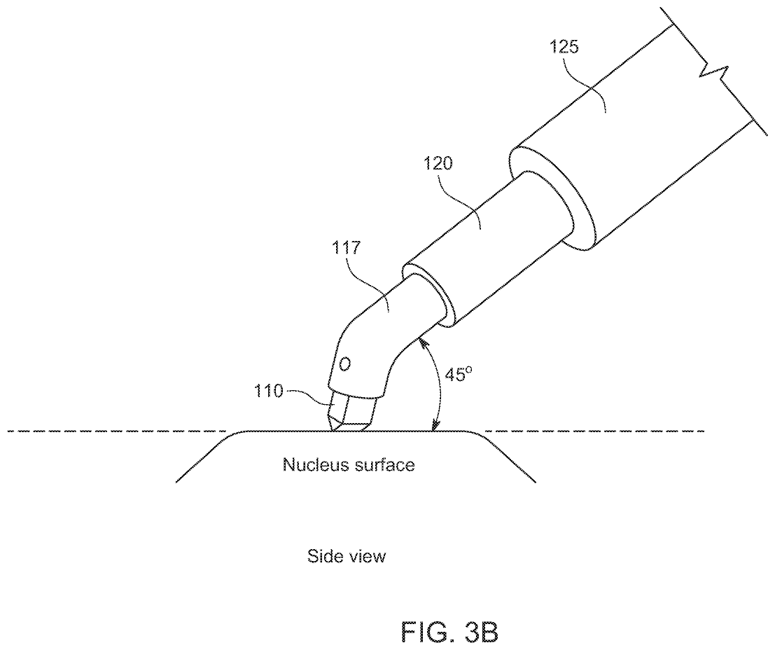

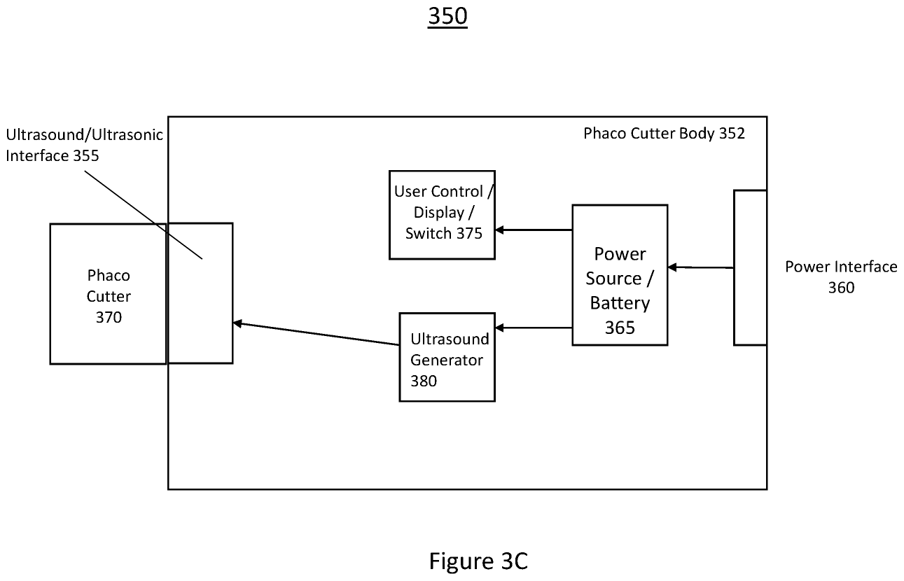

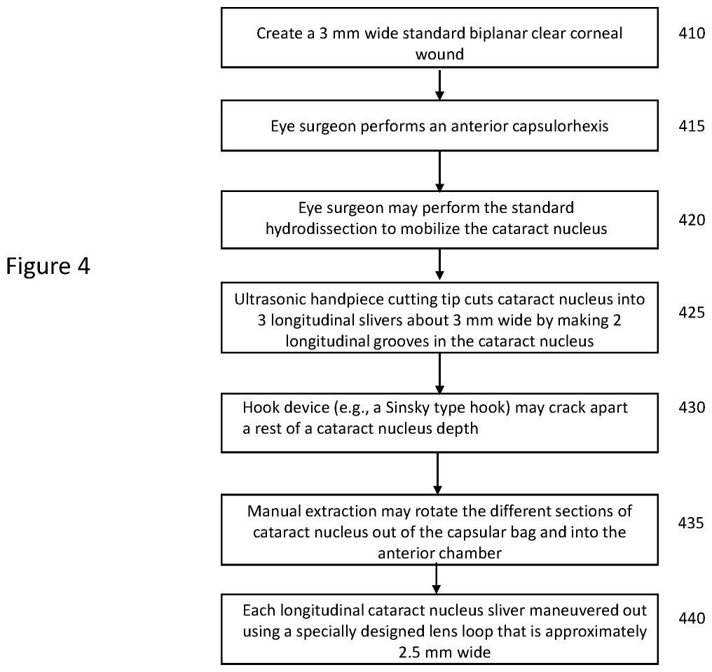

The Details: FIG. 4 illustrates a flowchart for performing the cataract surgical technique according to some embodiments. In some implementations, in step 410, an eye surgeon may create a 3 mm wide (or approximately 3 mm wide) standard biplanar clear corneal self-sealing wound as for a standard phaco procedure. FIG. 3A illustrates the created clear corneal wound according to some implementations.

In some implementations, in step 415, the eye surgeon performs an anterior capsulorhexis (with or without Trypan Blue depending on capsule visibility). In some implementations, in step 420, the eye surgeon may perform the standard hydrodissection to mobilize the cataract nucleus. In some implementations, in step 425, the improved portable ultrasonic handpiece with the unique phaco cutter tip may be used to cut the cataract nucleus into 3 longitudinal slivers each about 3 mm wide by making 2 longitudinal grooves in the cataract nucleus. In some embodiments, the 2 longitudinal grooves may be shown in FIG. 3A with cross-hatches or diagonal markings. In some implementations, the solid cutting blade tip may only be exposed beyond an irrigation sleeve of the phaco apparatus by 3 to 4 mm. In these embodiments, this limits a maximum groove depth of the longitudinal grooves to approximately 3 to 4 mm. FIG. 3A illustrates cutting the cataract nucleus into three longitudinal slivers according to some embodiments. In FIG. 3A, the cataract nucleus is reference number 318. In some embodiments, the portable ultrasonic cutting device 311 may include an irrigation sleeve 314 and/or a phaco cutter 310. In some embodiments, a phaco cutter 310 includes a phaco cutter body and a phaco cutter tip 316. In some embodiments, the phaco cutter tip 316 may be at a distal end of the phaco cutter 310. In the procedure or technique described herein, the phaco cutter tip 316 may create the two longitudinal grooves 317 and 325 in the cataract nucleus.

In some implementations, in step 430, a hook device (e.g., a Sinsky type hook) may crack apart the rest of the cataract nucleus depth utilizing a separate 1 mm corneal limbal incision 90 degrees away, which may be referred to as a standard chop maneuver.

In some implementations, in step 435, a manual rotation may be performed to bring the cataract nucleus out of the capsular bag after the ultrasonic cutting step has been performed and into the anterior chamber before the chop maneuver is performed. In these embodiments, a viscolastic is irrigated above and below the cataract nucleus to protect the cornea and the posterior capsule. Because the cataract nucleus is divided into 3 longitudinal slivers, in some implementation, in step 440, each longitudinal cataract nucleus sliver can be safely maneuvered out using a specially designed lens loop that is approximately 2.5 mm wide. In some implementations, the lens loop being approximately 2.5 mm wide may allow it to slip easily into the 3 mm entry wound and under the slivers of the nucleus. In this implementation, because the patient eye is pressurized with the viscolastic, the tendency is for the cataract nuclear slivers, once teased apart, to naturally flow out of the eye along with the excess viscolastic. In situations where viscolastic is not easily available an irrigating lens loop 2.5 mm wide can be used as an alternative to help float the slivers out of the eye.

In prior systems, an ultra chopper was utilized to chop the cataract nucleus into 4 quadrants for facilitating phaco emulsification. However, the ultra chopper still needs the expensive phaco machine. In that system, the ultra chopper's use is limited to chopping the nucleus into 4 quadrants for facilitating phaco emulsification. However, this Ultra chopper system still requires a phaco machine with all the expenses involved and requires medical professionals hooking up the ultra chopper. Then, this system requires setting up and tuning the standard Kelman phaco tip, then connecting the ultra chopper to do the nucleus chop, then changing back and resetting up the Kelman phaco tip to allow removal of the 4 nuclear quadrants using significant amounts of ultrasonic energy. This makes the process very cumbersome and time consuming, and still requires the full phaco machine and associated supplies thus and expense, hence the popularity of using this system has been limited.

In some embodiments, an inexpensive and unique cataract surgery pack may be created for performing cataract surgery. In some embodiments, the inexpensive and unique cataract surgery pack may include a portable rechargeable ultrasonic cutting handpiece for generating ultrasonic energy, one or more removable phaco cutters, and/or one or more irrigation sleeves. In addition, in some embodiments, the inexpensive cataract surgery pack may include a specially designed lens loop (which may be a 2.5 mm wide lens loop which is different from current lens loops which are 5 mm or wider and thus will not work). In some embodiments, the inexpensive cataract surgery pack may include a 1 mm spatula that may be inserted into a 1 mm entry wound 180 degrees away from the main entry wound to help push the cataract nucleus slivers out of the operative eye.

A significant advantage and benefit of the inexpensive cataract surgery pack may allow an inexpensive and less invasive procedure that utilizes a small self-sealing corneal incision and cuts the cataract nucleus into 3 equal or approximately equal slivers that are delivered out of the eye manually utilizing the tools described above. Accordingly, the expensive phaco machine does not need to be utilized. Further the risk to the patient is significantly reduced because a much lower amount of ultrasound energy is utilized to only cut the cataract nucleus, which reduces the risk of damaging the cornea.

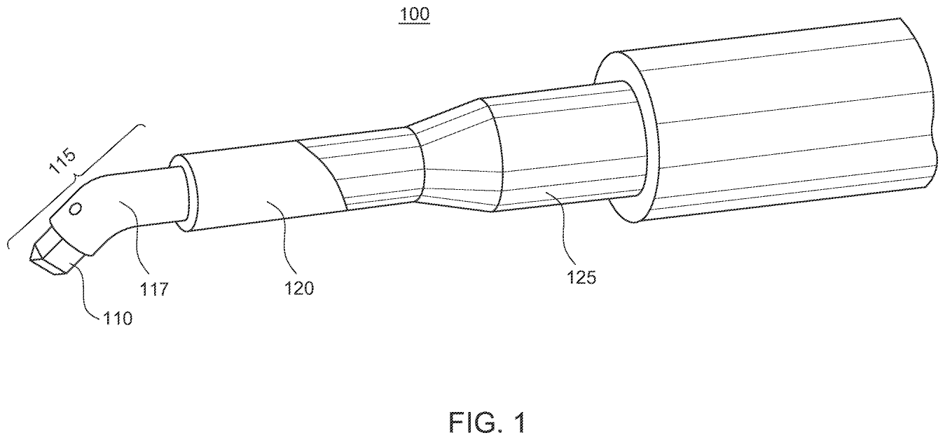

FIG. 1 illustrates a portable rechargeable ultrasonic cutting handpiece including a phaco cutter tip according to some embodiments. In some implementations, the portable rechargeable ultrasonic apparatus 100 may include an ultrasonic handpiece or body 125, an insulating sleeve 120, and a phaco cutter 115. In some implementations, the phaco cutter 115 may include a phaco cutter main body 117 and a phaco cutter tip 110. In some implementations, the phaco cutter tip 110 may be a distal end of the phaco cutter 115. In some implementations, a proximate end of the phaco cutter 115 may be inserted and/or screwed into a distal end of the ultrasonic headpiece 125. In some embodiments, the distal end of the ultrasonic handpiece 125 may have a circular hole into which a circular shaped proximal end of the phaco cutter 115 may be screwed in. In some embodiments, a silicone insulating irrigating sleeve 120 may cover a distal or far end of the ultrasonic handpiece 125. In some implementations, the phaco cutter tip 110 may have a width of 3 millimeters as shown by reference number 111. In some implementations, the width of the phaco cutter tip may range from 2.75 mm to 3.25 mm. In some implementations, a height of the phaco cutter tip 110 may be approximately 4 millimeters in height, or alternatively may range from in height from 3.5 millimeters to 4.5 millimeters in height. In some implementations, the phaco cutter tip may have a thickness of 0.3 millimeters, or alternatively the phaco cutter's tip 110 may have a thickness that ranges from 0.2 millimeters to 0.4 millimeters. In some embodiments, the phaco cutter body 117 may connect the phaco cutter tip 110 to the ultrasonic headpiece 125. In some embodiments, the phaco cutter body 117 may be constructed with a curve or bend to angle the phaco cutter tip 110 at a 45-degree angle with respect to the ultrasonic handpiece 125. In some embodiments, the ultrasonic handpiece 125 may be angled at approximately a 45-degree angle with respect to a surface of a cataract nucleus. FIG. 3B illustrates an ultrasonic cutting handpiece contacting a cataract nucleus according to some embodiments. In some embodiments, the phaco cutter tip 110 (or a distal end of the phaco cutter tip) may lie flat on a surface of the cataract nucleus. In some implementations, the phaco cutter body may be curved or bent to cause the phaco cutter tip 110 to be angled at a range from 35 to 50 degrees with respect to ultrasonic headpiece 125. This way even though the handpiece is held at a 45-degree angle, the blade of the phaco tip can be placed flat on the surface of the nucleus for maximum cutting efficiency.

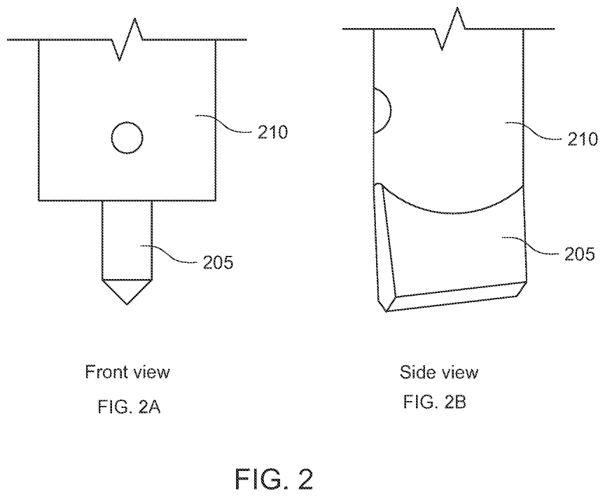

FIG. 2A illustrates a front view of the phaco cutter according to some embodiments. FIG. 2B illustrates a side view of the phaco cutter according to some embodiments. In FIG. 2, the width of the phaco cutter tip 205 may be 0.3 millimeters. In some embodiments, the width of the phaco cutter tip 205 may range from 0.2 millimeters and 0.4 millimeters. In some embodiments, as shown in FIG. 2A, the height of the phaco cutter tip 205 may be 4 millimeters in length. Alternatively, a height of the phaco cutter tip 205 may range from 3.5 millimeters to 4.5 millimeters. In some embodiments, as shown in FIG. 2A, the width of the phaco cutter body 210 may be 3 millimeters in width. Alternatively, in some embodiments, the width of the phaco cutter body 210 may range in width from 2 millimeters to 4 millimeters in width.

In some embodiments, the phaco cutter tip 210 may be placed or positioned squarely on the surface of the cataract nucleus and may gently dig downwards, using the entire 360 degree web of zonules for counter traction thus minimizing the stress on any particular segment of zonules. In these embodiments, gentle pressure is important and it is important to let the ultrasonic energy perform the digging step into the cataract rather than the downward pressure exerted on the phaco cutter tip 205. In these embodiments, the phaco cutter tip 205 cuts downwards due to the ultrasonic energy penetrating and emulsifying the cataract nuclear surface. In other words, the downwards pressure on the phaco cutter tip may only need to be enough to have secure contact between the surface of the phaco cutter tip 205 and the cataract nuclear surface to allow transfer of ultrasonic energy to the cataract nucleus. In these embodiments, the cutting action of the phaco cutter tip 205 itself does not require any downward force. Therefore, the cutting of the cataract nucleus is done by the ultrasonic energy and not by pushing or pulling the phaco cutter tip through the cataract nucleus with brute force. This allows this approach to be utilized even with weak zonules, or with a partial zonular dehiscence by just gently placing the phaco cutter tip 205 in the center on the surface of the cataract nucleus and letting the ultrasound energy do the actual cutting downwards.

In some embodiments, each trough may be the size of the phaco cutter tip 205 (e.g., 4 mm long, 0.3 mm wide and 3 to 4 mm deep). In these embodiments, normally 3 or 4 troughs adjacent to each other in a straight line will create a groove long enough and deep enough to allow the cataract nucleus to be divided with a phaco chop technique.

In a well dilated (7 to 8 mm) patient pupil, the phaco cutter tip may be placed on the nuclear surface in the center of the pupil with gentle pressure watching carefully for lens movement downwards, applying just enough pressure downwards to achieve good surface contact without pushing the lens backwards but just ever so slightly.

The phaco cutter tip 205 width of 0.3 mm may be selected because the narrower the width of the phaco cutter tip, the easier it is to cut the trough (less cataract nuclear surface to emulsify with the phaco cutter tip). In addition, a 0.3 mm wide groove is wide enough to allow for visualization of the base of the groove to be able to see if the groove is deep enough to ensure the cataract nucleus may break apart when it comes time to chop the cataract nucleus. In the Escaff ultra chopper embodiment, a thin knife-like phaco tip is used to create a ‘cut,’ not a groove. This is a problem because it is very hard to judge the depth of the cut as the two halves of the nucleus tend to stay apposed with no view of the base of the cut. In addition, such a thin cutting tip (knife-like) is too flimsy and does not give enough purchase on the inside wall of the groove to push the cataract nucleus halves apart easily when it comes time to do the chopping maneuver because the cataract nucleus has a tendency to either swivel apart still attached at the base plate of the nucleaus or the tip bends and slides over the surface of the cataract nucleus in an ineffective manner. In some embodiments, at the distal end of the phaco cutter tip, there is a bevel to allow phaco cutter tip to cut and/or dig into the cataract nucleus easier.

The height of the phaco cutter tip 205 may be selected to be 4 mm so that the groove created is deep enough so when it comes to chopping, the two adjacent portions of the cataract nucleus do not just swivel apart with the base plate of the cataract nucleus remaining unbroken. In some embodiments, in a hard cataract, it is very important to have complete separation of the two adjacent slivers of the cataract nucleus for later removal. If the two adjacent slivers of the cataract nucleus are still attached together at the base, this will make the removal of the slivers very difficult if not impossible.

FIG. 3C illustrates a block diagram of a portable rechargeable ultrasonic cutting device according to some embodiments. In some embodiments, the portable rechargeable ultrasonic cutting device 350 includes a cutting device body 352 and phaco cutter 370. In some embodiments, the phaco cutter 370 is connected to the cutting device body 352. In some embodiments, the cutting device body 352 may include a power interface 360, a battery or power device 365, a display or user interface 375, an ultrasound generating device 380 and/or an ultrasound contact or interface 355. In some embodiments, an external power source may be connected to the power interface 360 and may recharge the power source 365. In some embodiments, the power source 365 may be a rechargeable battery. In some embodiments, the power source 365 or battery may provide DC power to the display or user interface 375 or contact switch and/or the ultrasound generator 380. In some embodiments, the contact switch may be utilized to turn on or off the ultrasonic cutting device. In some embodiments, the ultrasound generator 380 may generate ultrasound energy and/or pulses and deliver the ultrasound energy to the ultrasound interface 355. In some embodiments, the ultrasound interface 355 is connected to the ultrasound generator device 380 and the phaco cutter 370 and delivers the generated ultrasound energy and/or pulses to the phaco cutter 370.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

Unless otherwise noted, the terms “connected to” and “coupled to” (and their derivatives), as used in the specification and claims, are to be construed as permitting both direct and indirect (i.e., via other elements or components) connection. In addition, the terms “a” or “an,” as used in the specification and claims, are to be construed as meaning “at least one of.” Finally, for ease of use, the terms “including” and “having” (and their derivatives), as used in the specification and claims, are interchangeable with and shall have the same meaning as the word “comprising”.

The processor as disclosed herein can be configured with instructions to perform any one or more steps of any method as disclosed herein.

As used herein, the term “or” is used inclusively to refer items in the alternative and in combination.

As used herein, characters such as numerals refer to like elements.

Embodiments of the present disclosure have been shown and described as set forth herein and are provided by way of example only. Anyone of ordinary skill in the art will recognize numerous adaptations, changes, variations and substitutions without departing from the scope of the present disclosure. Several alternatives and combinations of the embodiments disclosed herein may be utilized without departing from the scope of the present disclosure and the inventions disclosed herein. Therefore, the scope of the presently disclosed inventions shall be defined solely by the scope of the appended claims and the equivalents thereof.

1. A cataract surgery pack, comprising:
a portable rechargeable ultrasonic cutting handpiece for generating ultrasonic energy;
one or more removable phaco cutters;
one or more irrigation sleeves;
a lens loop; and
a spatula to be inserted into an entry wound to assist to push the cataract nucleus slivers out of the eye.

2. The cataract surgery pack of claim 1, wherein the lens loop is less than 2.5 millimeters wide.

3. The cataract surgery pack of claim 1, wherein the spatula is less than 1 millimeter wide.

4. A portable rechargeable ultrasonic apparatus to remove a cataract nucleus, comprising:
an ultrasonic handpiece or body;
an insulating sleeve, the insulating sleeve to cover a distal or far end of the ultrasonic handpiece or body; and
a phaco cutter to connect to the ultrasonic handpiece or body, the phaco cutter to include a phaco cutter main body and a phaco cutter tip, wherein the phaco cutter tip is disposed at the distal end of the phaco cutter and has a width ranging from 2.75 to 3.25 millimeters.

5. The portable rechargeable ultrasonic apparatus of claim 4, wherein the phaco cutter tip has a height ranging from 3.5 millimeters to 4.5 millimeters.

6. The portable rechargeable ultrasonic apparatus of claim 4, wherein the phaco cutter tip has a thickness ranging from 0.2 millimeters to 0.4 millimeters.

7. The portable rechargeable ultrasonic apparatus of claim 4, wherein the phaco cutter body may be shaped with a curve to angle the phaco cutter tip at a range of a 35-degree to 50-degree angle with respect to the ultrasonic handpiece.

8. The portable rechargeable ultrasonic apparatus of claim 7, wherein a distal end of the phaco cutter tip may lie flat on a surface of the cataract nucleus.

9. The portable rechargeable ultrasonic apparatus of claim 4, wherein the ultrasonic handpiece or body includes a power interface, a battery, a display, an ultrasound generating device and an ultrasound contact or interface.

10. The portable rechargeable ultrasonic apparatus of claim 9, further comprising an external power source, the external power source to connect to the power interface and to recharge the battery.

11. The portable rechargeable ultrasonic apparatus of claim 9, the battery to provide DC power to the display and the ultrasound generating device.

12. The portable rechargeable ultrasonic apparatus of claim 9, further comprising a contact switch, the contact switch to turn on or off the portable rechargeable ultrasonic apparatus.

13. The portable rechargeable ultrasonic apparatus of claim 9, wherein the ultrasound generating device to generate the ultrasonic energy and to transfer the ultrasound energy to the ultrasound interface.

14. The portable rechargeable ultrasonic apparatus of claim 13, wherein the ultrasound interface is coupled to the phaco cutter and provides the ultrasound energy to the phaco cutter.

15. A method for performing the cataract surgical technique, comprising:
creating a 3 mm wide standard biplanar clear corneal self-sealing wound;
performing an anterior capsulorhexis (with or without Trypan Blue);
performing the standard hydrodissection to mobilize the cataract nucleus;
using the unique phaco cutter tip to cut the cataract nucleus into 3 longitudinal slivers each about 3 mm wide by making 2 longitudinal grooves in the cataract nucleus;
cracking apart a remaining portion of the cataract nucleus depth utilizing Sinsky hook device;
performing a manual rotation to bring the cataract nucleus out of the capsular bag after the ultrasonic cutting step has been performed and into and anterior chamber; and
maneuvering or removing a number of longitudinal cataract nucleus slivers utilizing a lens loop.
